(19) United States
(12) Patent Application Publication
(10) Pub. No.: US 2026/0283675 A1
(43) Pub. Date: Sep. 24, 2026

(54) PULSE WAVE OPTIMIZATION TO MINIMIZE NEUROMUSCULAR STIMULATION DURING PULSED FIELD ABLATION TREATMENT

(71) Applicant: Medtronic, Inc., Minneapolis, MN (US)

(72) Inventors: Daniel C. Sigg, Saint Paul, MN (US); Damijan Miklavcic, Kranj (SI); Lars M. Mattison, Blaine, MN (US); Aleksandra Cvetkoska, Ljubljana (SI); Alenka Macek Lebar, Sentvid (SI); Matej Rebersek, Menges (SI)

(21) Appl. No.: 19/471,884

(22) PCT Filed: Apr. 5, 2024
(86) PCT No.: PCT/US2024/023330

§ 371 Date: Oct. 2, 2025

Related U.S. Application Data
(60) Provisional application No. 63/494,520, filed on Apr. 6, 2023.

(51) Int. Cl. A61B 18/12; A61B 18/00; A61B 18/14

(52) U.S. Cl. CPC A61B 18/1206; A61B 2018/00577; A61B 2018/00613; A61B 2018/0075; A61B 2018/00761; A61B 2018/1467; A61B 18/1492

(57) ABSTRACT

Methods and apparatus for performing medical ablation procedures. In an example, a medical treatment apparatus includes an ablation waveform generator and an electronic controller. The ablation waveform generator configured to apply electrical waveforms to a treatment element placeable in proximity to a treatment site of a patient. The electronic controller is configured to control the ablation waveform generator. The electronic controller is further configured to cause the ablation waveform generator to generate a biphasic waveform having an interphase delay and an interpulse delay greater than the interphase delay. The electronic controller is configured to cause the ablation waveform generator to apply the biphasic waveform to the treatment element.

### FIELD

This application relates generally to methods and systems associated with ablation catheters, ablation generators, and other medical devices and components used in various ablation procedures.

### BACKGROUND

The delivery of some types of ablation therapy involves the use of a reliable, powerful, and precisely controlled source of electrical energy, e.g., in the form of a high-voltage pulse generator or a radio-frequency (RF) generator. Electrical pulses or continuous wave (CW) sinusoids are delivered to the intended treatment sites to perform reversible or irreversible electroporation in the case of pulsed field ablation (PFA), and thermally induced necrosis via RF using an ablation-therapy delivery device.

### SUMMARY

Pulsed field ablation (PFA) treatment delivers electrical pulses to a treatment site by generating biphasic waveforms. Such waveforms are characterized by interphase delays (the delay between the positive and negative phases of a pulse, e.g., 204, 206, and d₁ in FIG. 2) and interpulse delays (the delay between pulses, e.g., d₂ in FIG. 2), pulse width (e.g., Tₚ of FIG. 2), and other parameters, such as cycle length, as well as rise and fall times. PFA is targeted to a treatment site, for example, the pulmonary veins of the left atrium or to an arrhythmogenic substrate in ventricle. While the region causing irreversible electroporation (IRE) is nearest the ablation electrodes, the electric fields generated by PFA treatment can extend significantly beyond the zone of IRE, leading to unwanted stimulation of neuromuscular structures. For example, pulsed fields can excite certain tissues including nerves and smooth and striated (e.g., skeletal) muscles, which can lead to patient discomfort and involuntary movement. This may disadvantageously require the use of deeper sedation during treatment. Some pulsed fields can also directly stimulate pain fibers (e.g., A-delta and/or C-fibers) leading to the corresponding pain/discomfort. Stimulation of vagal structures can result in bradycardia and cough reflex. The vagus nerve, also known as the tenth cranial nerve, cranial nerve X, or CN X, is a cranial nerve that interfaces with the parasympathetic control of the heart, lungs, and digestive tract. The vagus is the longest nerve of the autonomic nervous system in the human body and comprises both sensory and motor fibers. In addition, stimulation of smooth muscle cells and/or autonomic nerve innervating vessels can lead to vasoconstriction and possibly vasospasms, including vasospasms of the coronary arteries. Electric fields will also capture/stimulate cardiomyocytes (i.e., cardiac tissue) at a distance from the catheter (e.g., in region 304 of FIG. 3) potentially causing acute arrhythmia.

Where waveforms utilize relatively short interphase and interpulse delays, unwanted effects are reduced. However, the resulting higher frequency waveforms may generate excess heat and/or microbubbles. Increasing interphase and interpulse delays mitigates temperature increases, possibly coagulum and bubble formation. However, increasing interpulse delays can increase unwanted effects, in particular neuromuscular stimulation.

To address these problems, embodiments and aspects presented herein provide that the ablation waveform can be selected, modified, and/or adjusted such that adverse effects are reduced (e.g., minimized) while an intended therapeutic purpose is still achieved. Using the examples presented herein, waveforms are generated with increased interpulse delays and shorter (relative to the interpulse delays) interphase delays. Using such waveforms, embodiments presented herein apply PFA treatment, while reducing unwanted neuromuscular stimulation and patient discomfort.

Disclosed herein are, among other things, various aspects, features, and embodiments of methods and apparatus for performing various ablation procedures. One example provides a medical-treatment apparatus. The medical treatment apparatus includes an ablation waveform generator and an electronic controller. The ablation waveform generator configured to apply electrical waveforms to a treatment element placeable in proximity to a treatment site of a patient. The electronic controller is configured to control the ablation waveform generator. The electronic controller is further configured to cause the ablation waveform generator to generate a biphasic waveform having an interphase delay and an interpulse delay greater than the interphase delay. The electronic controller is configured to cause the ablation waveform generator to apply the biphasic waveform to the treatment element.

In some aspects, the techniques described herein relate to a medical treatment method, the method including: generating, with an ablation waveform generator, a biphasic waveform having an interphase delay and an interpulse delay greater than the interphase delay; and applying the biphasic waveform to a treatment element placeable in proximity to a treatment site of a patient.

In some aspects, the techniques described herein relate to a non-transitory computer-readable medium containing executable instructions that, when executed by one or more electronic processors, cause the one or more electronic processors to: generate, with an ablation waveform generator, a biphasic waveform having an interphase delay and an interpulse delay greater than the interphase delay; and apply the biphasic waveform to a treatment element placeable in proximity to a treatment site of a patient.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments, examples, aspects, and features of concepts that include the claimed subject matter and explain various principles and advantages of those embodiments, examples, aspects, and features.

FIG. 1A & FIG. 1B are block diagrams illustrating a medical system according to various examples.

FIG. 2 graphically illustrates an electrical waveform that can be delivered to a treatment site using the medical system of FIGS. 1A & 1B according to various examples.

FIG. 3 schematically illustrates regions in and around a treatment site of a patient during use of the medical system of FIGS. 1A & 1B according to various examples.

FIG. 4 is a table illustrating protocols that can be used to configure the medical system of FIGS. 1A & 1B according to various examples.

FIG. 5 & FIG. 6 graphically illustrate electrical waveforms that can be delivered to a treatment site using the protocols of FIG. 4 and the medical system of FIGS. 1A & 1B according to various examples.

FIG. 7A & FIG. 7B are bar charts illustrating aspects of the operation of the medical system of FIGS. 1A & 1B according to various examples.

FIG. 8 is a line graph illustrating aspects of the operation of the medical system of FIGS. 1A & 1B according to various examples.

FIG. 9A & FIG. 9B are bar charts illustrating aspects of the operation of the medical system of FIGS. 1A & 1B according to various examples.

FIG. 10 is a line graph illustrating aspects of the operation of the medical system of FIGS. 1A & 1B according to various examples.

FIG. 11 illustrates a flowchart of a method performed by the medical system of FIGS. 1A & 1B according to various examples.

FIG. 12A & FIG. 12B are bar charts illustrating aspects of the operation of the medical system of FIGS. 1A & 1B according to various examples.

FIG. 13 is a line graph illustrating aspects of the operation of the medical system of FIGS. 1A & 1B according to various examples.

FIG. 14 is a line graph illustrating aspects of the operation of the medical system of FIGS. 1A & 1B according to various examples.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of examples, aspects, and features illustrated.

In some instances, the apparatus and method components have been represented where appropriate by conventional symbols in the drawings, showing only those specific details that are pertinent to understanding the of various embodiments, examples, aspects, and features so as not to obscure the disclosure with details that will be readily apparent to those of ordinary skill in the art having the benefit of the description herein.

### DETAILED DESCRIPTION

For ease of description, some or all of the example systems presented herein are illustrated with a single exemplar of each of its component parts. Some examples may not describe or illustrate all components of the systems. Other example embodiments may include more or fewer of each of the illustrated components, may combine some components, or may include additional or alternative components.

It should be understood that although certain figures presented herein illustrate hardware and software located within particular devices, these depictions are for illustrative purposes only. In some embodiments, the illustrated components may be combined or divided into separate software, firmware, and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable communication links.

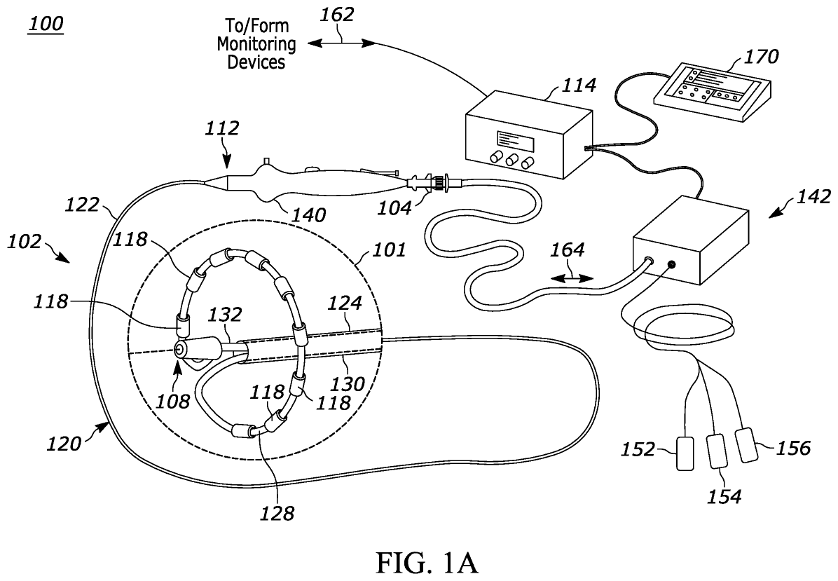

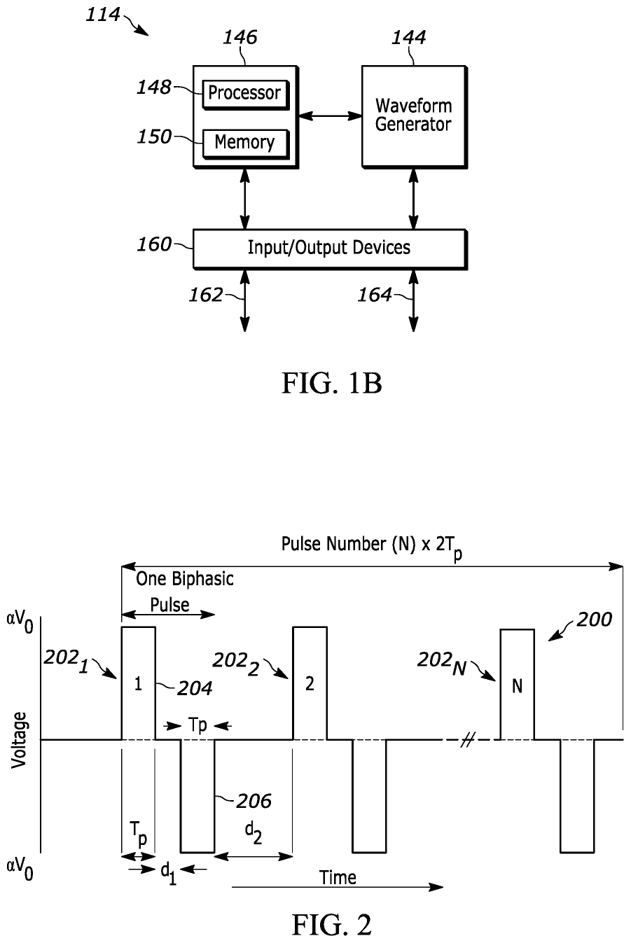

FIG. 1A and FIG. 1B are block diagrams illustrating a medical system 100 according to various examples. More specifically, FIG. 1A is a block diagram illustrating an overall view of the system 100 and FIG. 1B is a block diagram illustrating an electronic controller used in the system 100.

In a representative example, the system 100 includes a medical device 112 and the electronic controller 114 in communication with the medical device 112. In operation, the medical device 112 is used to deliver energy (for example, PFA or electroporation energy) for treating or ablating an area of target tissue. In various examples, the medical device 112 is also used to deliver one or more test pulses or waveforms to evaluate the correctness of its position or a potential for an adverse reaction caused by the delivery of such energy.

In the example shown, the medical device 112 includes a catheter 102, a magnified view of a portion 101 of which is shown in FIG. 1A. The catheter 102 has electrodes 118 for therapeutic interaction with the selected treatment site in or on the patient's body. In operation, the electrodes 118 deliver energy, for example, PFA energy, electroporation energy, test pulses and waveforms, and/or other transferred energy, to the treatment site. The catheter 102 includes an elongated body 120 passable through the patient's vasculature to enable placement of the electrodes 118 in proximity to the treatment site of a patient for diagnosis and/or treatment. The elongated body 120 has a proximal portion 122 and a distal portion 124 and typically includes one or more lumens that provide mechanical, electrical, and/or fluid communication between the proximal portion 122 and the distal portion 124. In some examples, the elongated body 120 has a central or guidewire lumen 130 hosting a shaft 132 and a carrier arm 128. The shaft 132 is longitudinally movable within and with respect to the guidewire lumen 130. In operation, longitudinal movement of the shaft 132 is used to cause the carrier arm 128 to transition between the first (e.g., substantially linear) configuration and a second (e.g., looped) configuration. As an illustration, FIG. 1A shows the carrier arm 128 in the looped configuration. The carrier arm 128 and the electrodes 118 may be referred to herein as a treatment element.

The medical device 112 also includes a handle 140 connected to the proximal portion 122 of the elongated body 120. In various examples, the handle 140 includes circuitry and structures for properly operating and manipulating the catheter 102. Additionally, the handle 140 typically includes one or more connectors 104 for electrically connecting the circuitry to the electronic controller 114, e.g., to establish electrical paths between various pertinent parts of the medical device 112 and pertinent components or parts of the electronic controller 114. The handle 140 also typically has one or more actuation or control features that enable the corresponding practitioner to control, deflect, steer, or otherwise manipulate the distal portion 124 via the proximal portion 122.

In some examples, the system 100 also includes a navigation system 142 used for guiding a medical treatment procedure. In the example shown, the medical device 112 is coupled to the electronic controller 114 through the navigation system 142. In some other examples, both the medical device 112 and the navigation system 142 are directly coupled to the electronic controller 114. The navigation system 142 is designed to help visualize the real-time position and orientation of the catheter 102 within the patient's body to increase the accuracy of targeted ablation and reacquisition of pacing sites for re-ablation. In a typical example, the navigation system 142 calculates the position and orientation of a catheter tip 108 using three magnetic sources 152, 154, 156 as references. The navigation system 142 typically relies on static magnetic fields that are calibrated and can be computer controlled. Due to the nature of magnetic fields, the orientation of the carrier arm 128 can be calculated even when the tip 108 is stationary. By calculating the strength and orientation of the magnetic fields at a given location, the Cartesian coordinates (x, y, z) of the tip 108 are typically calculated together with the roll, pitch, and yaw angles of the carrier arm 128.

In various examples, the electronic controller 114 includes components and circuits for the delivery of one or more energy modalities to the electrodes 118. In the example shown in FIG. 1B, the electronic controller 114 includes an ablation waveform generator 144, control circuitry 146, and input/output (I/O) devices 160. The control circuitry 146 includes a processing circuit (e.g., a general-purpose processor) 148 and a memory 150. The I/O devices 160 of the electronic controller 114 typically provide multiple I/O channels, e.g., including one or more channels 162 for communicating with the external monitoring devices, and at least one I/O channel 164 for operatively connecting the waveform generator 144 to the medical device 112 (also see FIG. 1A). The electronic controller 114 is operable in a plurality of operating modes, which can be selected for specific medical procedures as needed or appropriate.

In various examples, the memory 150 has buffers to temporarily store received data and nonvolatile data-storage devices to more permanently store data and program code. In operation, the memory 150 provides pertinent data and program code to the processing circuit 148. The program code, when executed by the processing circuit 148 enables the electronic controller 114 to perform signal processing and generate various control and communication signals. In some examples, the processing circuit 148 performs rendering processing of input signals received from the external monitoring devices and outputs, through the I/O devices 160, the corresponding viewable images, charts, and/or graphs for being viewed on an external display.

In various examples, the medical monitoring devices include one or more devices from the following group of devices: a diaphragmatic or thoracic excursion assessment device; an accelerometer; an electromyography (EMG) machine; an ECG recorder; a vital signs monitor; an airway pressure monitor; and an expiratory carbon-dioxide monitor. In other examples, other suitable patient monitoring devices are similarly used and linked to the electronic controller 114 through the I/O devices 160. In some examples, the I/O devices 160 include at least one I/O interface device for supporting a wireless data link.

In some examples, the electronic controller 114 is also connected, via the I/O devices 160, to an operator interface device 170. In various examples, the operator interface device 170 includes: a touch screen; buttons, knobs, and/or keys; light indicators (e.g., colored light emitting diodes, LEDs); a speaker; a switch; a joystick; and other control accessories connected and configured to enable the operator to properly operate the electronic controller 114 and the medical device 112. The operator interface device 170 also typically enables the operator to see thereon at least some indicators generated by the electronic controller 114 in response to the signals received via the I/O devices 160 from the medical monitoring devices.

FIG. 2 an electrical waveform 200, which can be delivered to the treatment site using the system 100 according to various examples. The electrical waveform 200 comprises a sequence of N biphasic (bipolar) pulses 202, which are labeled 202₁, 202₂, . . . , 202_{N}, respectively. Each of the biphasic pulse 202 includes a positive pulse 204 and a negative pulse 206. Both the positive pulse 204 and the negative pulse 206 have the amplitude αV₀ and the pulse width Tₚ, where α is the scaling factor (e.g., in the range between 0.1 and 10). The time delay between the positive pulse 204 and the negative pulse 206 is d₁. The parameter d₁ referred to herein as the interphase delay. The time delay between two consecutive biphasic pulses 202 in the waveform 200 is d₂. The parameter d₂ is referred to herein as the interpulse delay. The amplitude V₀, the pulse width Tₚ, and the parameters α, N, d₁, and d₂ of the waveform 200 are selectable and controllable via the waveform generator 144.

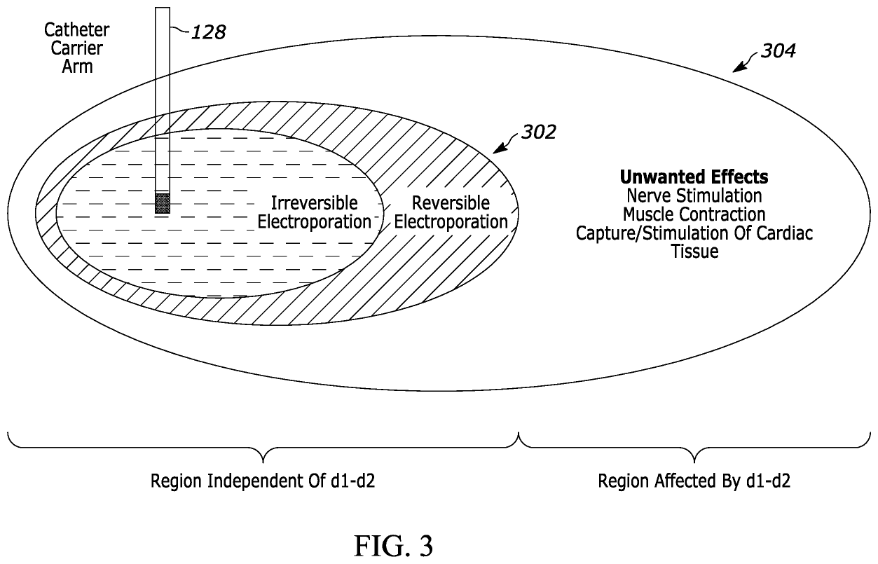

FIG. 3 schematically illustrates regions of a patient during treatment with the medical system 100. In particular, FIG. 3 illustrates regions of effect relative to the placement the carrier arm 128 of the catheter 102. In region 302, both irreversible electroporation and reversible electroporation of tissue occurs. In region 304, neuromuscular stimulation and other unwanted effects of the application of PFA waveforms (e.g., the waveform 200 of FIG. 2) occurs, as described herein. Irreversible and reversible electroporation occurs in region 302 independent of the values for d₁ and d₂ of the applied waveform. However, the occurrence of unwanted effects is affected by the values for d₁ and d₂ of the applied waveform. The area and/or volume of nerve excitation extends beyond the volume of cell membrane permeabilization due to lower threshold of the transmembrane voltage (TMV).

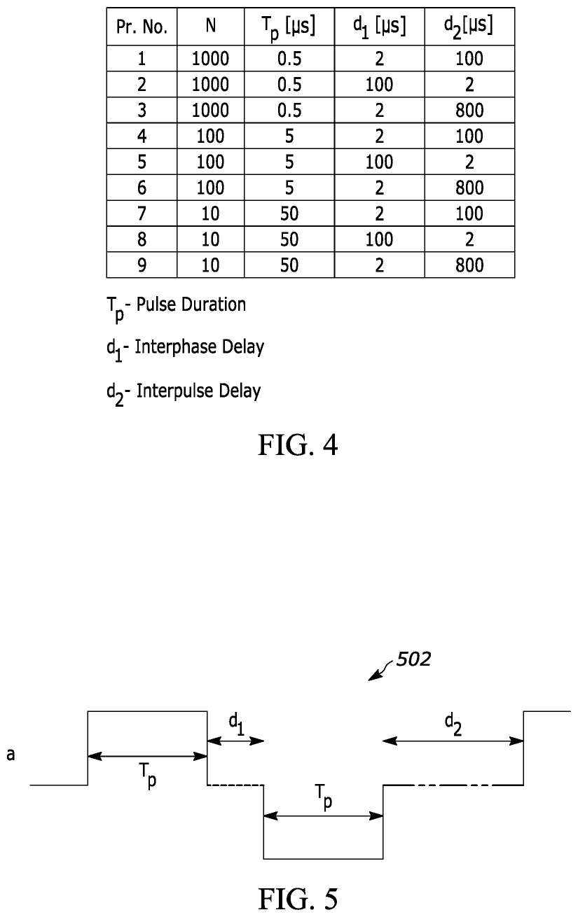

As described below with respect to FIGS. 4-10, the unwanted effects that can accompany PFA treatment may be lessened without impacting the effectiveness of the desired treatment (i.e., the levels of irreversible and reversible electroporation) by selecting values for d₁ and d₂.

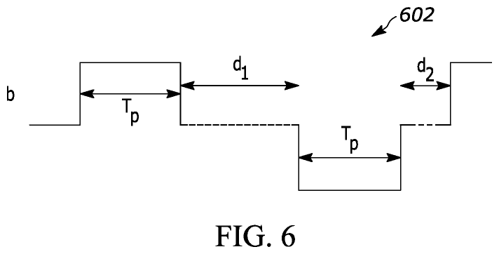

FIG. 4 is a table illustrating a number of protocols, each representing a different variation of the waveform 200. As shown in FIG. 4, each protocol defines its waveform with values for Tₚ, d₁, d₂, and N, which are the values of the pulse parameters for the biphasic pulse protocols. All protocols have equal total on-time, i.e., N×2Tₚ=1 ms. Tₚ specifies the pulse width (equal for positive and negative phase), N is number of pulses, d₁ specifies the interphase delay, and d₂ specifies the interpulse delay. As illustrated, the values of Tₚ and N vary for each group of three protocols (1-3, 4-6, 7-9), but are the same within the group. Protocols 1, 3, 4, 6, 7, and 9 specify a waveform where the interphase delay (d₁) is lesser than the interpulse delay (d₂). FIG. 5 illustrates an example waveform 502 of this type. Protocols 2, 5, and 8 specify a waveform where the interphase delay (d₁) is greater than the interpulse delay (d₂). FIG. 6 illustrates an example waveform 602 of this type.

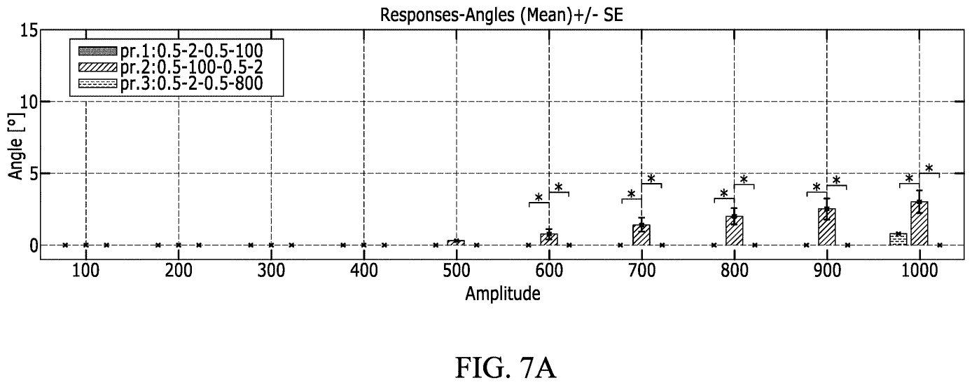

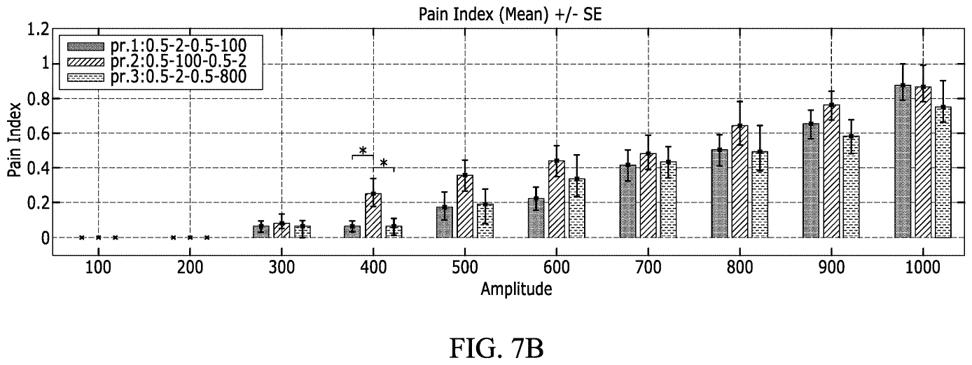

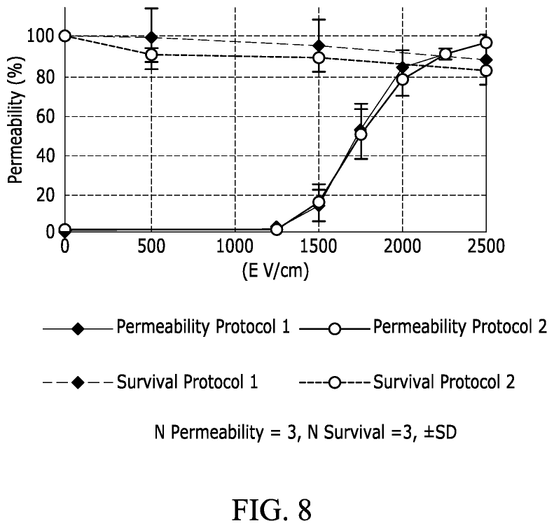

FIGS. 7A, 7B, and 8 compare the results for PFA treatment applied using protocols 1, 2, and 3, using, for example, the medical system 100. As shown in FIG. 4, the value of Tₚ for protocols 1-3 is 0.5 μs and the value of N is 1000. In protocol 1, the value of the interphase delay (d₁) is 2 μs and the value of the interpulse delay (d₂) is 100 μs. In protocol 2, the value of the interphase delay (d₁) is 100 μs and the value of the interpulse delay (d₂) is 2 μs. In protocol 3, the value of the interphase delay (d₁) is 2 μs and the value of the interpulse delay (d₂) is 800 μs. FIG. 7A is a bar chart illustrating unwanted neuromuscular contraction resulting from the application of the waveforms described by protocols 1-3. As can be seen in FIG. 7A, the incidence of unwanted neuromuscular contraction is lower for protocols 1 and 3 across a range of amplitudes. FIG. 7B is a bar chart illustrating pain indices (as reported by the patient) resulting from the application of the waveforms described by protocols 1-3. As can be seen in FIG. 7B, the pain indices are lower for protocols 1 and 3 across a range of amplitudes. FIG. 8 is a line graph showing the percentages for permeability (i.e., reversible electroporation) and survival (i.e., irreversible electroporation) in region 302 (see FIG. 3) as caused by waveforms applied according to protocols 1 and 2. As illustrated in FIG. 8, the differences in permeability and survival between protocols 1 and 2 is essentially the same.

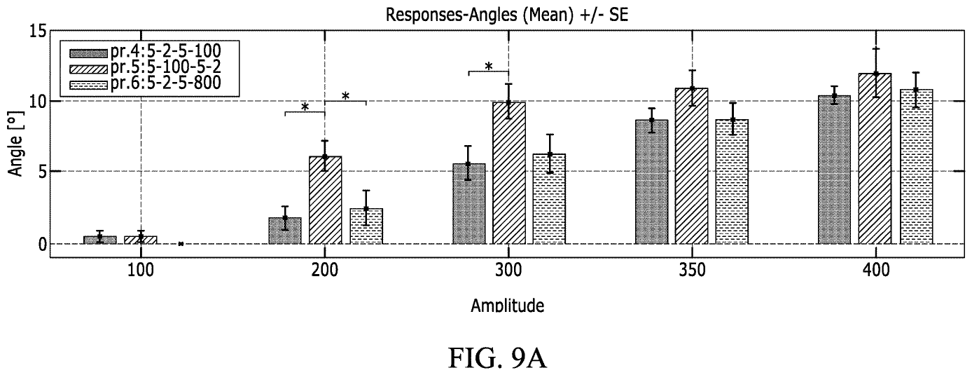

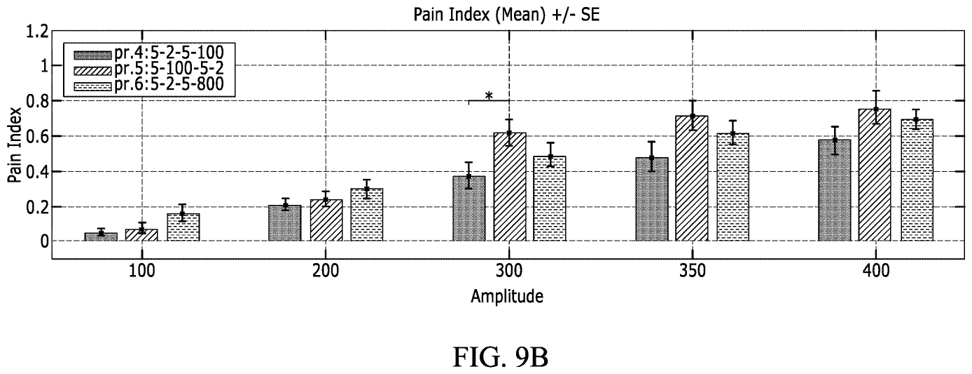

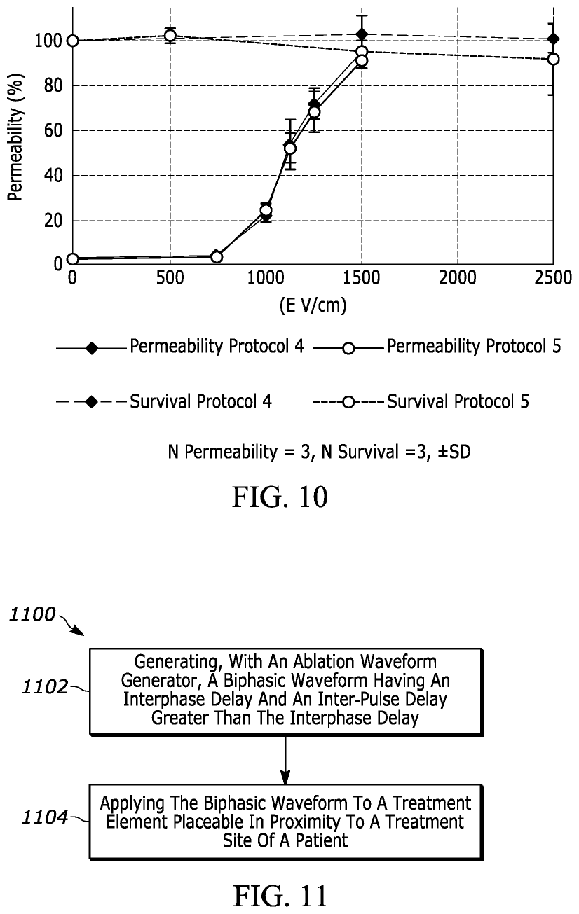

FIGS. 9A, 9B, and 10 compare the results PFA treatment applied using protocols 4, 5, and 6, using, for example, the medical system 100. As shown in FIG. 4, the value of Tₚ for protocols 4-6 is 5 μs and the value of N is 100. In protocol 4, the value of the interphase delay (d₁) is 2 μs and the value of the interpulse delay (d₂) is 100 μs. In protocol 5, the value of the interphase delay (d₁) is 100 μs and the value of the interpulse delay (d₂) is 2 μs. In protocol 6, the value of the interphase delay (d₁) is 2 μs and the value of the interpulse delay (d₂) is 800 μs. FIG. 9A is a bar chart illustrating unwanted neuromuscular contraction resulting from the application of the waveforms described by protocols 4-6. As can be seen in FIG. 9A, the incidence of unwanted neuromuscular contraction is lower for protocols 4 and 6 than for protocol 5 across a range of amplitudes. FIG. 9B is a bar chart illustrating pain indices (as reported by the patient) resulting from the application of the waveforms described by protocols 4-6. As can be seen in FIG. 9B, the pain indices are lower for protocols 4 and 6 than for protocol 5 across a range of amplitudes. FIG. 10 is a line graph showing the percentages for permeability (i.e., irreversible electroporation) and survival (i.e., reversible electroporation) in region 302 (see FIG. 3) as caused by waveforms applied according to protocols 4 and 5. As illustrated in FIG. 10, the differences in permeability and survival between protocols 4 and 5 is essentially the same.

FIG. 11 is a flowchart illustrating the method 1100 for performing pulsed field ablation treatment according to various examples. In some examples, the method 1100 is implemented via the electronic controller 114 in a fully automatic mode, in which no additional input from the system's human operator is needed after the method is initiated. In some other examples, the method 1100 has one or more checkpoints, wherein a human decision or prompt is solicited. In such examples, the remainder of the method 1100 is still executed by the corresponding controller circuitry of the electronic controller 114 in an automated manner, e.g., by running a corresponding program code.

The method 1100 includes generating, with an ablation waveform generator, a biphasic waveform having an interphase delay and an interpulse delay greater than the interphase delay (in block 1102). In some examples, the electronic controller 114 causes the ablation waveform generator 144 to generate a waveform similar to that illustrated in FIG. 2. In some examples, the ablation waveform generator 144 generates the waveform 200 using parameters specified in FIG. 4 for protocols 1, 4, or 7. In some examples, the ratio between the interpulse delay and the interphase delay is 50:1. In some examples, the pulse width for the waveform remains constant regardless of the values for the interpulse delay and the interphase delay.

The method 1100 includes applying electrical waveforms to a treatment element placeable in proximity to a treatment site of a patient (in block 1104). In some examples, the electronic controller 114 causes the ablation waveform generator 144 to apply the biphasic waveform 200 to the electrodes 118 of the carrier arm 128 to apply the electrical waveforms the treatment site of the patient.

In some alternative examples, the electronic controller 114 causes the ablation waveform generator 144 to generate a waveform where the interphase delay and the interpulse delay modulate over the course of the PFA treatment. In some alternative examples, the electronic controller 114 causes the ablation waveform generator 144 to generate a waveform where the interphase delay and the interpulse delay modulate in relation to the pulse width. In some alternative examples, the electronic controller 114 causes the ablation waveform generator 144 to generate a waveform where the interphase delay and the interpulse delay modulate in relation to amplitude. In some alternative examples, the electronic controller 114 causes the ablation waveform generator 144 to generate a waveform where the interphase delay and the interpulse delay modulate in response to patient discomfort. In some alternative examples, the electronic controller 114 causes the ablation waveform generator 144 to generate a waveform where the interphase delay and the interpulse delay are set prior to treatment through the use of a short subtherapeutic pulse to establish patient sensitivity.

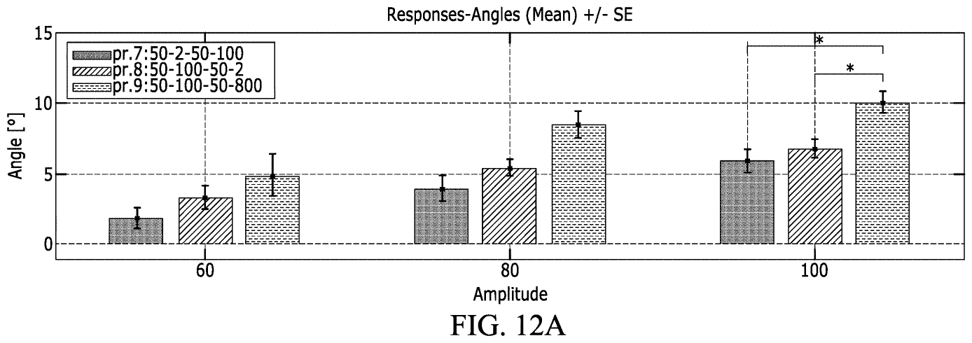

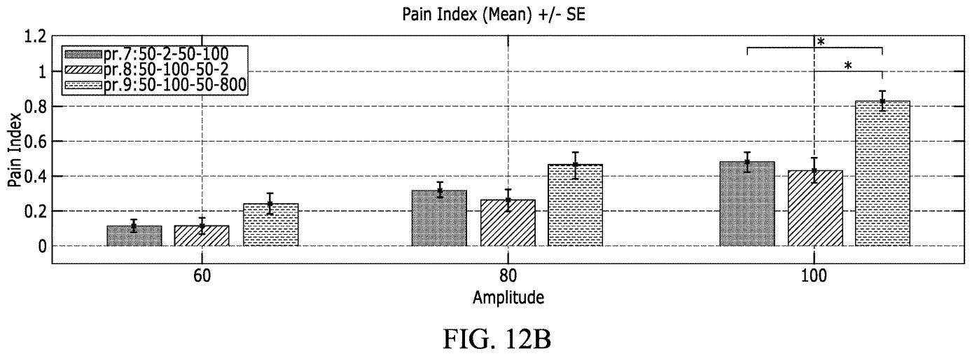

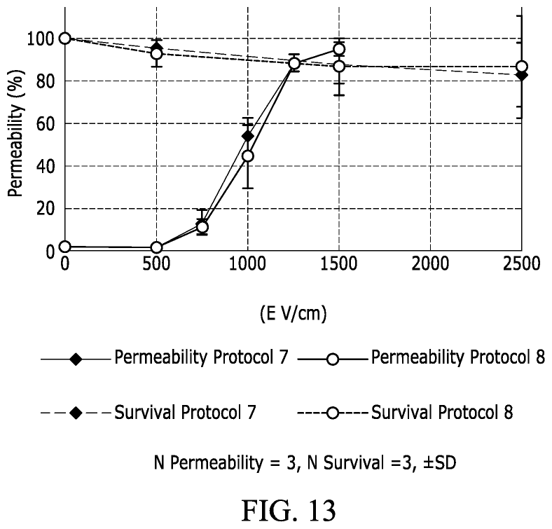

FIGS. 12A, 12B, and 13 compare the results PFA treatment applied using protocols 7, 8, and 9, using, for example, the medical system 100. As shown in FIG. 4, the value of Tₚ for protocols 7-9 is 5 μs and the value of N is 10. In protocol 7, the value of the interphase delay (d₁) is 2 μs and the value of the interpulse delay (d₂) is 100 μs. In protocol 8, the value of the interphase delay (d₁) is 100 μs and the value of the interpulse delay (d₂) is 2 μs. In protocol 9, the value of the interphase delay (d₁) is 100 μs and the value of the interpulse delay (d₂) is 800 μs. FIG. 12A is a bar chart illustrating unwanted neuromuscular contraction resulting from the application of the waveforms described by protocols 7-9. As can be seen in FIG. 12A, the incidence of unwanted neuromuscular contraction is lower for protocol 7 than for protocols 8 and 9 across a range of amplitudes. FIG. 12B is a bar chart illustrating pain indices (as reported by the patient) resulting from the application of the waveforms described by protocols 7-9. As can be seen in FIG. 12B, the pain indices are lower for protocol 8 than for protocols 7 and 9 across a range of amplitudes. FIG. 13 is a line graph showing the percentages for permeability (i.e., irreversible electroporation) and survival (i.e., reversible electroporation) in region 302 (see FIG. 3) as caused by waveforms applied according to protocols 7 and 8. As illustrated in FIG. 10, the differences in permeability and survival between protocols 7 and 9 is essentially the same.

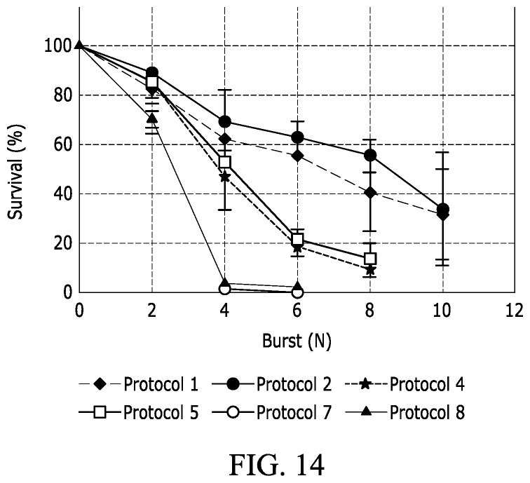

FIG. 14 is a graph illustrating increasing the burst number for protocols 1, 2, 4, 5, 7, and 8 for a cell lines CHO. The results are shown as the mean value of three repetitions (each point)±standard deviation (vertical bars).

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain implementations and should in no way be construed to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” et cetera, should be read to recite one or more of the indicated elements unless a claim recites an explicit limitation to the contrary.

Unless explicitly stated otherwise, each numerical value and range should be interpreted as being approximate as if the word “about” or “approximately” preceded the value or range.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

Unless otherwise specified herein, in addition to its plain meaning, the conjunction “if” may also or alternatively be construed to mean “when” or “upon” or “in response to determining” or “in response to detecting,” which construal may depend on the corresponding specific context. For example, the phrase “if it is determined” or “if [a stated condition] is detected” may be construed to mean “upon determining” or “in response to determining” or “upon detecting [the stated condition or event]” or “in response to detecting [the stated condition or event].”

Also, for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” et cetera, imply the absence of such additional elements. The same type of distinction applies to the use of terms “attached” and “directly attached,” as applied to a description of a physical structure. For example, a relatively thin layer of adhesive or other suitable binder can be used to implement such “direct attachment” of the two corresponding components in such physical structure.

The described embodiments are to be considered in all respects as only illustrative and not restrictive. In particular, the scope of the disclosure is indicated by the appended claims rather than by the description and figures herein. All changes that come within the meaning and range of equivalency of the claims are to be embraced within their scope.

The functions of the various elements shown in the figures, including any functional blocks labeled as “processors” and/or “controllers,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and nonvolatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

As used in this application, the term “circuitry” may refer to one or more or all of the following: (a) hardware-only circuit implementations (such as implementations in only analog and/or digital circuitry); (b) combinations of hardware circuits and software, such as (as applicable): (i) a combination of analog and/or digital hardware circuit(s) with software/firmware and (ii) any portions of hardware processor(s) with software (including digital signal processor(s)), software, and memory(ies) that work together to cause an apparatus, such as a mobile phone or server, to perform various functions); and (c) hardware circuit(s) and or processor(s), such as a microprocessor(s) or a portion of a microprocessor(s), that requires software (e.g., firmware) for operation, but the software may not be present when it is not needed for operation.” This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

It should be appreciated by those of ordinary skill in the art that any block diagrams herein represent conceptual views of illustrative circuitry embodying the principles of the disclosure. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

The following paragraphs provide various examples of the embodiments disclosed herein.
Example 1. A medical treatment apparatus, comprising: an ablation waveform generator configured to apply electrical waveforms to a treatment element placeable in proximity to a treatment site of a patient; and an electronic controller configured to control the ablation waveform generator, the electronic controller being further configured to: cause the ablation waveform generator to generate a biphasic waveform having an interphase delay, and an interpulse delay greater than the interphase delay; and cause the ablation waveform generator to apply the biphasic waveform to the treatment element.
Example 2. The medical treatment apparatus of example 1, wherein the electronic controller is further configured to: cause the ablation waveform generator to generate the biphasic waveform wherein the ratio of the interpulse delay to the interphase delay is in a range of 50:1 to 1000:1.
Example 3. The medical treatment apparatus of example 1, wherein the electronic controller is further configured to: cause the ablation waveform generator to generate the biphasic waveform wherein the ratio of the interpulse delay to the interphase delay is 50:1.
Example 4. The medical treatment apparatus of any of examples 1-3, wherein the value of the interphase delay is two microseconds, and the value of the interpulse delay is one-hundred microseconds.
Example 5. The medical treatment apparatus of any of examples 1-4, wherein the electronic controller is further configured to: cause the ablation waveform generator to generate the biphasic waveform having a pulse width, wherein the pulse width remains constant despite the values for the interpulse delay and the interphase delay.
Example 6. The apparatus of any of examples 1-5, wherein the apparatus is configured to perform a medical procedure selected from the group of procedures consisting of pulsed field ablation, radio-frequency ablation, reversible electroporation, and irreversible electroporation.
Example 7. A medical treatment method, the method comprising: generating, with an ablation waveform generator, a biphasic waveform having an interphase delay and an interpulse delay greater than the interphase delay; and applying the biphasic waveform to a treatment element placeable in proximity to a treatment site of a patient.
Example 8. The medical treatment method of example 7, further comprising: generating the biphasic waveform, wherein the ratio of the interpulse delay to the interphase delay is in a range of 50:1 to 1000:1.
Example 9. The medical treatment method of example 7, further comprising: generating the biphasic waveform, wherein the ratio of the interpulse delay to the interphase delay is 50:1.
Example 10. The medical treatment method of any of examples 7-9, wherein the value of the interphase delay is two microseconds, and the value of the interpulse delay is one-hundred microseconds.
Example 11. The medical treatment method of any of examples 7-10, further comprising: generating the biphasic waveform having a pulse width, wherein the pulse width remains constant despite the values for the interpulse delay and the interphase delay.
Example 12. The medical treatment method of any of examples 7-11, further comprising: performing a medical procedure selected from the group of procedures consisting of pulsed field ablation, radio-frequency ablation, reversible electroporation, and irreversible electroporation.
Example 13. A non-transitory computer-readable medium containing executable instructions that, when executed by one or more electronic processors, cause the one or more electronic processors to: generate, with an ablation waveform generator, a biphasic waveform having an interphase delay and an interpulse delay greater than the interphase delay; and apply the biphasic waveform to a treatment element placeable in proximity to a treatment site of a patient.
Example 14. The non-transitory computer-readable medium of example 13, wherein the executable instructions further cause the one or more electronic processors to: generate the biphasic waveform, wherein the ratio of the interpulse delay to the interphase delay is in a range of 50:1 to 1000:1.
Example 15. The non-transitory computer-readable medium of example 13, wherein the executable instructions further cause the one or more electronic processors to: generating the biphasic waveform, wherein the ratio of the interpulse delay to the interphase delay is 50:1.
Example 16. The non-transitory computer-readable medium of any of examples 13-15, wherein the value of the interphase delay is two microseconds, and the value of the interpulse delay is one-hundred microseconds.
Example 17. The non-transitory computer-readable medium of any of examples 13-16, wherein the executable instructions further cause the one or more electronic processors to: generate the biphasic waveform having a pulse width, wherein the pulse width remains constant despite the values for the interpulse delay and the interphase delay.
Example 18. The non-transitory computer-readable medium of any of examples 13-17, wherein the executable instructions further cause the one or more electronic processors to: control one or more medical devices to perform a medical procedure selected from the group of procedures consisting of pulsed field ablation, radio-frequency ablation, reversible electroporation, and irreversible electroporation.

1. A medical treatment apparatus, comprising:
an ablation waveform generator configured to apply electrical waveforms to a treatment element placeable in proximity to a treatment site of a patient; and
an electronic controller configured to control the ablation waveform generator, the electronic controller being further configured to:
cause the ablation waveform generator to generate a biphasic waveform having an interphase delay, and an interpulse delay greater than the interphase delay; and
cause the ablation waveform generator to apply the biphasic waveform to the treatment element.

2. The medical treatment apparatus of claim 1, wherein the electronic controller is further configured to:
cause the ablation waveform generator to generate the biphasic waveform wherein a ratio of the interpulse delay to the interphase delay is in a range of 50:1 to 1000:1.

3. The medical treatment apparatus of claim 1, wherein the electronic controller is further configured to:
cause the ablation waveform generator to generate the biphasic waveform wherein a ratio of the interpulse delay to the interphase delay is 50:1.

4. The medical treatment apparatus of claim 1, wherein the value of the interphase delay is two microseconds, and the value of the interpulse delay is one-hundred microseconds.

5. The medical treatment apparatus of claim 1, wherein the electronic controller is further configured to:
cause the ablation waveform generator to generate the biphasic waveform having a pulse width, wherein the pulse width remains constant despite the values for the interpulse delay and the interphase delay.

6. The apparatus of claim 1, wherein the apparatus is configured to perform a medical procedure selected from a group of procedures consisting of pulsed field ablation, radio-frequency ablation, reversible electroporation, and irreversible electroporation.

7. A medical treatment method, the method comprising:
generating, with an ablation waveform generator, a biphasic waveform having an interphase delay and an interpulse delay greater than the interphase delay; and
applying the biphasic waveform to a treatment element placeable in proximity to a treatment site of a patient.

8. The medical treatment method of claim 7, further comprising:
generating the biphasic waveform, wherein a ratio of the interpulse delay to the interphase delay is in a range of 50:1 to 1000:1.

9. The medical treatment method of claim 7, further comprising:
generating the biphasic waveform, wherein a ratio of the interpulse delay to the interphase delay is 50:1.

10. The medical treatment method of claim 7, wherein the value of the interphase delay is two microseconds, and the value of the interpulse delay is one-hundred microseconds.

11. The medical treatment method of claim 7, further comprising:
generating the biphasic waveform having a pulse width, wherein the pulse width remains constant despite the values for the interpulse delay and the interphase delay.

12. The medical treatment method of claim 7, further comprising:
performing a medical procedure selected from a group of procedures consisting of pulsed field ablation, radio-frequency ablation, reversible electroporation, and irreversible electroporation.

13. A non-transitory computer-readable medium containing executable instructions that, when executed by one or more electronic processors, cause the one or more electronic processors to:
generate, with an ablation waveform generator, a biphasic waveform having an interphase delay and an interpulse delay greater than the interphase delay; and
apply the biphasic waveform to a treatment element placeable in proximity to a treatment site of a patient.

14. The non-transitory computer-readable medium of claim 13, wherein the executable instructions further cause the one or more electronic processors to:
generate the biphasic waveform, wherein a ratio of the interpulse delay to the interphase delay is in a range of 50:1 to 1000:1.

15. The non-transitory computer-readable medium of claim 13, wherein the executable instructions further cause the one or more electronic processors to:
generating the biphasic waveform, wherein a ratio of the interpulse delay to the interphase delay is 50:1.

16. The non-transitory computer-readable medium of claim 13, wherein the value of the interphase delay is two microseconds, and the value of the interpulse delay is one-hundred microseconds.

17. The non-transitory computer-readable medium of claim 13, wherein the executable instructions further cause the one or more electronic processors to:
generate the biphasic waveform having a pulse width, wherein the pulse width remains constant despite the values for the interpulse delay and the interphase delay.

18. The non-transitory computer-readable medium of any of claim 13, wherein the executable instructions further cause the one or more electronic processors to:
control one or more medical devices to perform a medical procedure selected from a group of procedures consisting of pulsed field ablation, radio-frequency ablation, reversible electroporation, and irreversible electroporation.
